(19) United States
(12) Patent Application Publication
(10) Pub. No.: US 2005/0054088 A1
(43) Pub. Date: Mar. 10, 2005

(54) Method of separating plasma membrane proteins

(76) Inventors: Amir Mikel Rahbar, Silver Spring, MD (US); Catherine Fenselau, Baltimore, MD (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/701,062

(22) Filed: Nov. 5, 2003

Related U.S. Application Data
(60) Provisional application No. 60/424,054, filed on Nov. 5, 2002.

(51) Int. Cl. G06F 19/00; G01N 33/48; G01N 33/50

(52) U.S. Cl. 435/317.1; 702/19; 530/383

(57) ABSTRACT

Plasma membranes proteins are separated with a colloidal silica plasma membrane, and processed for identification by mass spectrometry and bioinformatics.

### CROSS-REFERENCE TO RELATED APPLICATIONS

This application claims the benefit of U.S. Provisional Application No. 60/424,054, filed Nov. 5, 2002. The subject matter of U.S. Provisional Application No. 60/424,054, is hereby incorporated by reference herein in its entirety.

### BACKGROUND OF THE INVENTION

(1) Field of the Invention

The invention relates to separating of proteins, and more particularly to separating plasma membrane proteins.

(2) Description of Related Art

Drug discovery is an important field of research in the pharmaceutical and biotechnology industry. Plasma membranes are rich in drug targets and other proteins responsible for cell signaling, transport, signal transduction, and other cellular functions. Information obtained about these proteins helps to facilitate the drug discovery process. Although these proteins play an important role in cellular function, they are usually expressed in very low abundance and are therefore hard to identify and analyze. In order to be able to identify the proteins in the plasma membrane it is important to start out with a plasma membrane fraction that is free of contamination from other more abundant proteins from the cell. 2D gel electrophoresis remains to be the primary tool for use with proteomics and drug discovery, however, the inability of membrane proteins to be separated using isoelectric focusing excludes 2D gel electrophoresis as a viable technique of plasma membrane protein separation.

With the completion of the sequencing of the human genome and recent advances in mass spectrometry, proteomics has become an increasingly important field of biological research. Proteomics can be defined as the analysis of the protein complement of an organism or biological system at a given point in time, under a specific set of conditions.

While genomic analysis of mRNA levels can give you information on what proteins may be expressed in a biological system, they do not always reflect the actual expression levels of the proteins they encode. The information obtained using proteomics can be used for qualitative and quantitative analysis, so that it can be determined what proteins are being expressed and determine the relative expression levels of the protein as well.

More than 50% of current drug targets belong to a class of proteins called membrane proteins. ( Drug discovery: a historical perspective.Drews J. Science. 2000 Mar 17;287(5460):1960-4.) (J. Drews and S. Ryser. eds. Human Disease-From Genetic Causes to Biochemical Effects. Berlin: Blackwell Science, 1997.) Although about 30% of proteins in the cell are thought to be embedded in the various cell membranes, there is less known about this protein class than any other class of protein in the cell. Structures of membrane proteins are hard to obtain using X-ray crystallography and NMR due to their hydrophobic nature. This is because of their inability to form crystals and their insolubility in solutions without using detergents and organic solvents. Because of this, only 2% of the structures in the Protein Data Bank are classified as membrane proteins (Kyogoku Y, Fujiyoshi Y, Shimada I, Nakamura H, Tsukihara T, Akutsu H, Odahara T, Okada T, Nomura N. Structural genomics of membrane proteins. Acc Chem Res. 2003 Mar;36(3):199-206.)

Although membrane proteins can make up as much as 30% of the total protein in a cell, plasma membrane proteins make up only from 2-5% of the total cellular protein, and with this in mind, it is important to start with a subcellular fraction that is highly enriched for plasma membrane proteins. According to Henderson's Dictionary of Biological Terms, the plasma membrane is defined as the “membrane bounding the surface of all living cells formed of a fluid lipid belayed in which proteins carrying out the functions of enzymes, ion pumps, transport proteins, receptors for hormones etc. are embedded. It regulates the entry and exit of most solutes and ions, few substances being able to diffuse through unaided.” (Henderson's Dictionary of Biological Terms, 11^{th} edition, ref found at http://2 12.187.1 55.84/wnv/subdirectories_for_search/glossary&references_contents/bo okscontents/b73.htm)

Isolating a total membrane fraction is simple, but separating membrane fractions into their individual organelles is much harder. The isolation of the subcellular plasma membrane fraction is in particular quite difficult due to its low percentage of total cellular protein, its similarity to other organelle membrane components, and its propensity to exist in different structures such as open sheets or membrane vesicles. For these and other reasons, it has historically been difficult for researchers to get an enriched plasma membrane fraction with a high degree of purity for study.

Mass spectrometry has become the most critical tool in proteomic research today. Data obtained from the mass spectrometry analysis of peptides and proteins, combined with bioinformatics using protein and genomic databases, is used for identification and analysis of proteins. Protein identification of whole proteins from prokaryotic organisms is possible using the calculated protein masses from genomic sequence data. This is because prokaryotic organisms do not extensively modify their proteins after expression. Eukaryotic organisms, on the other hand, cannot be analyzed in this fashion due in large part to the large amount of post translational modifications, which alter the experimental masses of the proteins from the masses predicted by the genomic data.

When determining the proteome of a particular cell line or tissue type, usually the first step is to fractionate the cell into its individual components, or organelles. (Schirmer, E., Gerace, L. Organellar Proteomics: the Prizes and Piffalls of Opening the Nuclear Envelope. Genome Biolog. 2002 Mar 13; 3(4): 1008.1-1008.4.)

These components include the cytosol, the nucleus, the mitochondria, the plasma membrane, the Golgi complex, the smooth endoplasmic reticulum, and the rough endoplasmic reticulum. This reduces the complexity of the system, which will increase the likelihood of identifying proteins that are present in lower copy numbers.

Isolation, characterization, and identification of membrane proteins have historically posed problems for scientists. Subcellular fractionation of cellular membranes is often tedious, time consuming, and can result in large losses of starting material. (Chaney, L., Jacobson, B. Coating cells with colloidal silica for high yield isolation of plasma membrane sheets and identification of transmembrane proteins. i JBiol Chem. Aug. 25, 1983; 258(16):10062-72.) It is also common for organellular cross-contamination to occur during the subcellular fractionation process, (Schirmer, E., Gerace, L. Organellar Proteomics: the Prizes and Piffalls of Opening the Nuclear Envelope. Genome Biolog. Mar. 13, 2002; 3(4): 1008.1-1008.4.) which will decrease the reliability of any comparative proteomics study.

In order to conduct a comparative study, one has to be reasonably sure that one has a representative sample of the system under study, not just parts of an organelle which may contain microenvironments of particular proteins while excluding other parts which may have been lost during the subcellular fractionation process. Once the desired fraction is obtained, the solubilization and identification of membrane proteins can be hindered by the highly hydrophobic nature of these proteins. Membrane proteins do not behave well once they are removed from the membrane. They are hydrophobic and will precipitate and aggregate in aqueous environments. Traditional proteomic strategies do not work with membrane proteins. 2D gel electrophoresis can separate complex protein mixtures with high resolution but excludes large proteins, hydrophobic proteins, and proteins with extreme PI's. (Santoni, V., Molloy, M., Rabilloud, T. Membrane proteins and proteomics: un amour impossible? Electrophoresis. 2000 April; 21(6): 1054-1070.) Membrane proteins are hydrophobic and are usually large, so 2D gel electrophoresis cannot be used.

The most common way to identify eukaryotic proteins is to create an environment where the proteins can be digested with some proteolytic enzyme, followed by analysis of the peptides by mass spectrometry. One way to do this is called the “shotgun” approach. A complex mixture of proteins is digested with some proteolytic enzyme, most commonly trypsin, and the peptides are then separated by liquid chromatography and analyzed by mass spectrometry. This technique, however, is limited to soluble proteins due to the incompatibility of membrane protein solubilization techniques with enzymatic digestion.

Another common technique is to separate a complex protein mixture using 2D gel electrophoresis before protein digestion. The proteins are first separated by their isoelectric points, then by their molecular weights. The unsurpassed resolution of 2D gels makes this a good way of separating complex protein mixtures. Once the proteins are separated, the protein spots can be excised from the gel, digested with trypsin, and its peptides analyzed by mass spectrometry for further protein identification and analysis. This technique is however not without its limitations. Membrane proteins are underrepresented in 2D gels due to their incompatibility with isoelectric focusing. For this reason, membrane proteins are often separated by 1D SDS-PAGE. Although the resolution of proteins on 1D gels is much worse than on 2D gels, it can still provide a crude separation of a complex protein mixture, while still providing an environment in which the membrane proteins may by digested with proteolytic enzymes. For this reason it is critical to start with a membrane fraction that is as pure as possible.

### BRIEF SUMMARY OF THE INVENTION

In some embodiments, this invention is directed to a method of separating plasma membranes, comprising the steps of: isolating plasma membranes with a cationic colloidal silica plasma membrane isolation step; washing the isolated plasma membranes with sodium carbonate to produce silica coated plasma membrane; prior to any proteolytic digestion, solubilizing the silica coated plasma membrane in a solubilizing agent, for example 2% SDS; and separating the plasma membrane proteins on a gel, for example an SDS PAGE gel.

In some embodiments, this invention further comprises excising the proteins; subjecting the proteins to “in gel” proteolytic enzyme digestion, for example trypsin digestion; and then analyzing the proteins with mass spectrometry.

In some embodiments, this invention is directed to a method of separating plasma membranes, comprising the steps of: isolating plasma membranes; washing the isolated plasma membranes; solubilizing the silica coated plasma membrane; and separating the plasma membrane proteins. In some embodiments, this invention further comprises excising the proteins; subjecting the proteins to “in gel” trypsin digestion; and then analyzing the proteins with mass spectrometry. In some embodiments, said washing of the isolated plasma membranes is conducted with sodium carbonate. In some embodiments, the solubilizing of the silica coated plasma membrane is conducted in 2% SDS. In some embodiments, the plasma membrane proteins are separated with a cationic colloidal silica plasma membrane. In some embodiments, the solubilizing of the silica coated plasma membrane is conducted prior to any proteolytic digestion. In some embodiments, cells are coated in suspension. In some embodiments cells are coated while adhering to a surface, e.g. that on which they are grown.

### BRIEF DESCRIPTION OF THE SEVERAL VIEWS OF THE DRAWINGS

Embodiments of this invention will now be described in detail with reference to the attached Figures, in which:

FIG. 1 illustrates plasma membrane isolation for RPMI 8226 multiple myeloma, in accordance with embodiments of this invention;

FIG. 2 illustrates a centrifugation step in accordance with embodiments of this invention

FIG. 3 illustrates plasma membrane isolation from MCF7 breast cancer cells in accordance with embodiments of this invention;

FIG. 4 illustrates an experimental scheme in accordance with embodiments of this invention;

FIG. 5 shows a western blot for determination of plasma membrane enrichment in accordance with embodiments of this invention;

FIG. 6 is a table showing plasma membrane enrichment for various cell lines in accordance with embodiments of this invention;

FIG. 7 is a table showing plasma membrane proteins identified from RPMI 8226 cells in accordance with embodiments of this invention;

FIG. 8 illustrates results obtained for RPMI 8226 multiple myeloma cells in accordance with embodiments of this invention;

FIG. 9 is a table showing basolateral plasma membrane proteins identified from MXR MCF-7 cells in accordance with embodiments of this invention;

FIG. 10 illustrates results obtained for MCF-7 breast cancer cells in accordance with embodiments of this invention; and

FIG. 11 shows a schematic outline of an embodiment of the present approach.

### DETAILED DESCRIPTION OF THE INVENTION

This invention provides the identification of plasma membrane proteins and evidence towards their topological position inside the membrane based on the identification of the peptides cleaved from the cytosolic domains of plasma membrane proteins. This selective cleavage of the cytosolic domains allows for the identification of proteins that were previously unavailable for proteolytic digestion.

When doing membrane proteomics there are two main problems that need to be overcome: how to obtain a purified fraction of the subcellular organelle of interest, and once it is, how to determine what proteins are present.

In some embodiments, this invention combines the cationic colloidal silica plasma membrane isolation technique (Cells have a slight negative charge on their exterior. Positively charged silica microbeads are attached to the plasma membrane covering the exterior of the cell. The positively charged silica coating is then coated with polyacrylic acid of a molecular weight of around 100,000 Da. This process increases the density of the plasma membrane making it easier to separate from the rest of the cellular components by centrifugation over density gradient material) (Chaney, L., Jacobson, B. Coating cells with colloidal silica for high yield isolation of plasma membrane sheets and identification of transmembrane proteins. JBiol Chem. 1983 Aug. 25; 258(16):10062-72.) with selective digestion of the cytosolic domains of plasma membrane proteins (Rahbar, A., Fenselau, C. Identification of Plasma Membrane Proteins: Use of a Modified Colloidal Silica Plasma Membrane Isolation Technique combined with Proteolysis and LC-MS/MS. Presented at the Proteome Society Meeting: Proteomics: New Techniques and Novel Technology, University of Maryland, College Park, Md., Nov. 5, 2002.), tandem mass spectrometry, (Dancik, V., Addona, T., Clauser, K., Vath, J., Pevzner, P. De novo peptide sequencing via tandem mass spectrometry. J Comput Biol. 1999 Fall-Winter; 6(3-4): 327-42.) and protein database searching to identify plasma membrane proteins and determine their topology in the plasma membrane. (Mascot Search Engine. (http://www.matrixscience.com); Swiss-Prot Protein Knowledge Database. (http://www.expasy.org/sprot/)).

Using a modified form of the cationic colloidal silica technique for plasma membrane isolation, methods in accordance with this invention have been developed that result in a highly purified plasma membrane fraction which can then be separated on a gel, for example on an SDS-PAGE gel, for identification and analysis by mass spectrometry.

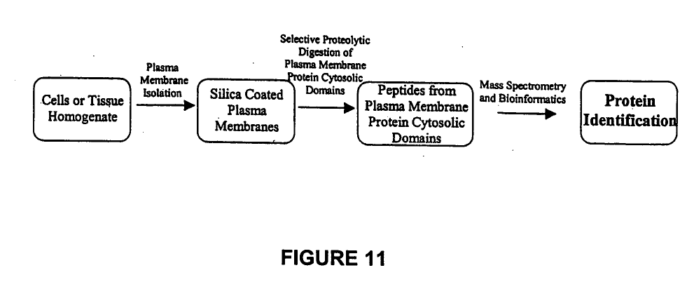

Mammalian cell plasma membranes are isolated. Other patents disclose use of a similar technique to isolate the plasma membrane as a first step in the isolation of caveolae, which are specialized lipid rafts that perform a number of signaling functions (See U.S. Pat. Nos. 6,255,457; 5,914,127; and 5,776,770), and Caprion Pharmaceuticals Inc. uses this plasma membrane isolation technique as a part of its proprietary subcellular proteomics platform called CellCarta™. FIG. 11 shows an outline of an embodiment of the present approach.

In embodiments of this invention, cells are harvested and resuspended in, for example, plasma membrane coating buffer (for example 20-50 mM MES, preferably 20 mM MES (MES is just a buffer to maintain the pH, 20 mM is sufficient); 140-160 mM NaCl, preferably about 150 mM NaCl. This amount of NaCl is sufficient to mimic the salinity of physiological conditions like in the buffer PBS. Too little NaCl and the cells swell, too much and they shrink); and 200-800 mM Sorbitol (Sorbitol is added to the coating buffer to add osmotic pressure to the cells to help them burst when placed in the lysis buffer)). The cell suspension is added drop wise to a suitable solution of cationic colloidal silica, for example a 1-20% solution of cationic colloidal silica and preferably a 10% solution of cationic colloidal silica, in plasma membrane coating buffer and left at a suitable low temperature, for example 0-10° C. and preferably about 4° C., with rotation for a suitable period of time, for example 5-60 min, preferably about 15 min. After the excess silica is washed away, the silica coated cell pellet is resuspended in, for example, plasma membrane coating buffer and added drop wise to a solution such as a solution of polyacrylic acid, preferably to a 10 mg/ml solution of polyacrylic acid (average molecular weight of ˜100 KDa), in plasma membrane coating buffer and left at a suitable low temperature, for example 0-10° C. and preferably about 4° C., with rotation for a suitable period of time, for example 5-60 min, preferably about 15 min. The excess polyacrylic acid is washed away and the cells are resuspended in a lysis buffer (for example, 1-10 MM Imadazole and preferably 2.5 mM Imidazole pH 7.0 with protease inhibitors) for 30-60 min on ice. During this time the cells can be agitated every 2-10 minutes, preferably about every 5 minutes or so, to keep the cells from settling to the bottom (before being resuspended in the lysis buffer, the coating process may be repeated several times to increase the thickness of the coating around the cells if needed). The cells are lysed (for examples N₂ cavitation, Dounce homogenizer, etc) and the cell homogenate is centrifuged for a suitable period of time, for example about 5-20 minutes and preferably about 10 minutes at 100-2000 g, preferably about 900 g, to sediment the silica coated plasma membranes and nuclei. The silica coated plasma membranes and nuclei are resuspended in lysis buffer and run over a gradient, for example Nycodenz and preferably 70% Nycodenz, in lysis buffer and centrifuged, for example at 10,000 to 40,000 g for 10 to 60 minutes and preferably at about 28,000 g for about 40 minutes. The nuclei stop at the interface between the gradient and the lysis buffer and the silica coated plasma membranes readily sediment through the gradient and pellet at the bottom. The supernatant is drawn off and the silica coated plasma membranes are resuspended in lysis buffer and washed a plurality of times, for example 2-5 times, and preferably 3 times, with the same lysis buffer.

The silica coated plasma membranes are then placed in a suitable solution of salts and chaotropic agents for a suitable period of time such as 30 min to 2 hours, and preferably about 1 hour (for example in 5-300 mM and preferably 150 mM Na₂CO₃+0.5-2M and preferably 1M NaCl, or 2-8 M and preferably 4M Urea+0.5-2M and preferably 1M NaCl) to remove the peripheral membrane proteins and any other proteins that may have been isolated along with the plasma membrane. While the Plasma membranes are still in the denaturing agents the disulfide bonds can be reduced and alkylated. The silica coated plasma membranes are centrifuged to separate the plasma membranes from the peripheral proteins, washed a suitable number of times, for example 2-5 times and preferably 3 times in a suitable wash solution, for example PBS, washed a suitable number of times, for example 2-6 times and preferably 3 times in another suitable wash solution, for example 25-100 mM and preferably 50 mM ammonium bicarbonate with a pH of about 7.8, and placed directly in a solution of proteolytic enzyme, for example and preferably trypsin. Since the exterior of the plasma membrane are covered by the silica and the enzymes do not cleave proteins on the membrane interior, only the cytosolic domains of the plasma membrane proteins are digested. The undigested proteins, still immobilized in the plasma membranes by the silica, are centrifuged away, and the remaining peptides are analyzed by tandem mass spectrometry.

The data from the mass spectrometry analysis are entered into a protein database search engine to identify the proteins in the plasma membrane. Membrane protein primary sequences can be entered into a computer that can generate a hydropathy plot to calculate the transmembrane regions of the plasma membrane protein. Since only the peptides from the cytosolic domains of the plasma membrane proteins are digested and identified, the membrane protein topology can be determined when compared to where they are in the protein primary sequence.

A list of the plasma membrane proteins identified with this method is listed in Table I. The strength of the method described here is that portions of the plasma membrane proteins can be digested without removing them from their lipid environment. This avoids the problems associated with the aggregation and precipitation of plasma membrane proteins when they are removed from the plasma membrane. The method described here is fast, inexpensive and a representative sample of the plasma membrane can be isolated in a relatively high yield, allowing for isotope incorporation for later quantitation. The technique allows for the proteolytic digestion of proteins that were previously unavailable for enzymatic digestion. Finally, the method can determine the topology of the plasma membrane proteins by comparing the peptides identified with the primary sequence of the membrane protein they came from, which is much faster and cheaper than using affinity and genetic techniques to determine topology.

TABLE I

| | Molecular weight |
|---|---|
| Integral Plasma Membrane Proteins Identified | |
| Sodium channel protein, brain I alpha subunit | 231 |
| Cystic fibrosis transmembrane conductance regulator | 169 |
| Potential phospholipid-transporting ATPase VC | 131 |
| Plasma membrane calcium-transporting ATPase 3 | 125 |
| Plasma membrane calcium-transporting ATPase 2 | 137 |
| ATP-binding cassette, sub-family A, member 1 | 256 |
| Complement receptor type 2 precursor (Cr2) | 116 |
| Voltage-gated potassium channel HERG | 128 |
| Metabotropic glutamate receptor 3 precursor | 100 |
| Myoferlin | 235 |
| Toll-like receptor 9 precursor | 111 |
| Peripheral Plasma Membrane Proteins Identified | |
| Spectrin beta chain, brain 4 | 419 |
| Polycystin (PKD 1) | 468 |
| Apical-like protein (APXL) | 178 |
| Golgi autoantigen | 261 |
| Epiplakin | 555 |
| Myosin | 224 |
| Neurofibromin | 325 |
| Plectin 1 | 520 |
| Actin cross-linking family protein 7 | 617 |
| Ankyrin 2 | 433 |
| Transgelin 2 | 23 |
| Phosphatidylethanolamine-binding protein | 21 |
| Calcium-Signal modulating cyclophilin | 33 |
| ligand | |

Additionally, in some embodiments of this invention, by omitting the proteolytic digestion step and solubilizing the silica coated plasma membrane directly, for example, in 2% SDS, the proteins can be separated on, for example, an SDS PAGE gel. The proteins can then be excised from the gel and subjected, for example, to “in gel” trypsin digestion, followed by analysis by mass spectrometry. Using this method, 48 total proteins, 21 of which were previously determined to be located in the plasma membrane, were identified. 44% of the proteins identified from the RPMI 8226 cell line were plasma membrane proteins.

This method was then applied to MCF-7 breast cancer adherent cell cultures. This previously published method coats the exposed cell surface plasma membrane, but not the part of the plasma membrane attached to the cell culture flask. After cell lysis, the plasma membranes not covered in the silica remain attached to the cell culture flask. By performing a series of washes with NaCl, EDTA, and sodium carbonate, a highly purified plasma membrane fraction was obtained. By solubilizing the plasma membranes in 2% SDS, the plasma membrane proteins were separated on an SDS PAGE gel to identify them as before. Using this technique, 23 plasma membrane proteins were identified from a total of 42 proteins identified from the MCF-7 cell line, so 55% of the proteins identified were previously identified as plasma membrane proteins.

The addition of the wash steps after the plasma membrane isolation allows a highly enriched plasma membrane fraction to be obtained. Because these fractions are relatively free of contamination from other cellular proteins, the plasma membrane proteins can then be identified from an SDS PAGE gel using “in gel” proteolytic digestion and mass spectrometry. This method can work with both suspension cell cultures and adherent cell cultures.

Thus, using a modified from of the cationic colloidal silica plasma membrane isolation technique, pure fractions of plasma membrane are obtained that are highly enriched with plasma membrane proteins. Using this fraction, a proteomic analysis of the plasma membrane can be conducted from both suspension cell cultures and adherent cell culture monolayers. An example in accordance with embodiments of this invention is described in detail below.

### EXAMPLE

Materials

The Criterion precast gel system, Mini-PROTEAN 3 electrophoresis system, Mini trans-blot cell, Criterion precast gels (13.3×8.7 cm, 12.5%, 8-16%, and 4-15%), Biosafe Coomassie stain, 10× PBS, 10× Tris/Glycine/SDS Buffer, 10× Tris/Glycine Buffer, pre-stained protein broad range standards, Laemmli sample buffer, nitrocellulose, and filter paper were purchased from Bio-Rad. MgCl₂, MES, NaCl, LUDOX-CL cationic colloidal silica, polyacrylic acid (100,000 typical molecular weight), imidazole, Nycodenz, Na₂CO₃, EDTA disodium salt, protease inhibitor cocktail, ProteoQwestä Colorimetric Western Blotting Kit, human α-Na/K ATPase primary antibody, DTT, lodoacetamide, fetal calf serum, and TFA were purchased from Sigma Aldrich. NH₄HCO₃, formic acid, acetonitrile, acetic acid, methanol, and CaCl₂ were purchased from Fisher. C18 ZipTips were purchased from Millipore. Modified porcine trypsin was purchased from Promega. Improved Minimal Essential Medium was purchased from ATCC. NanoES Spray capillaries were purchased from Protana.

Cell Culture

Human breast cancer mitoxantrone resistant cell line MCF-7 (MXR MCF-7) and human multiple myeloma cell line RPMI 8226 were used. MXR MCF-7 cells were grown in Improved Minimal Essential Medium containing 5% fetal calf serum and antibiotics at 37° C. and 5% CO₂. RPMI 8226 cells were grown in the same fashion but using RPMI 1640 media and heat inactivated fetal calf serum.

Preparation of Plasma Membrane Fraction from Suspension Cell Cultures

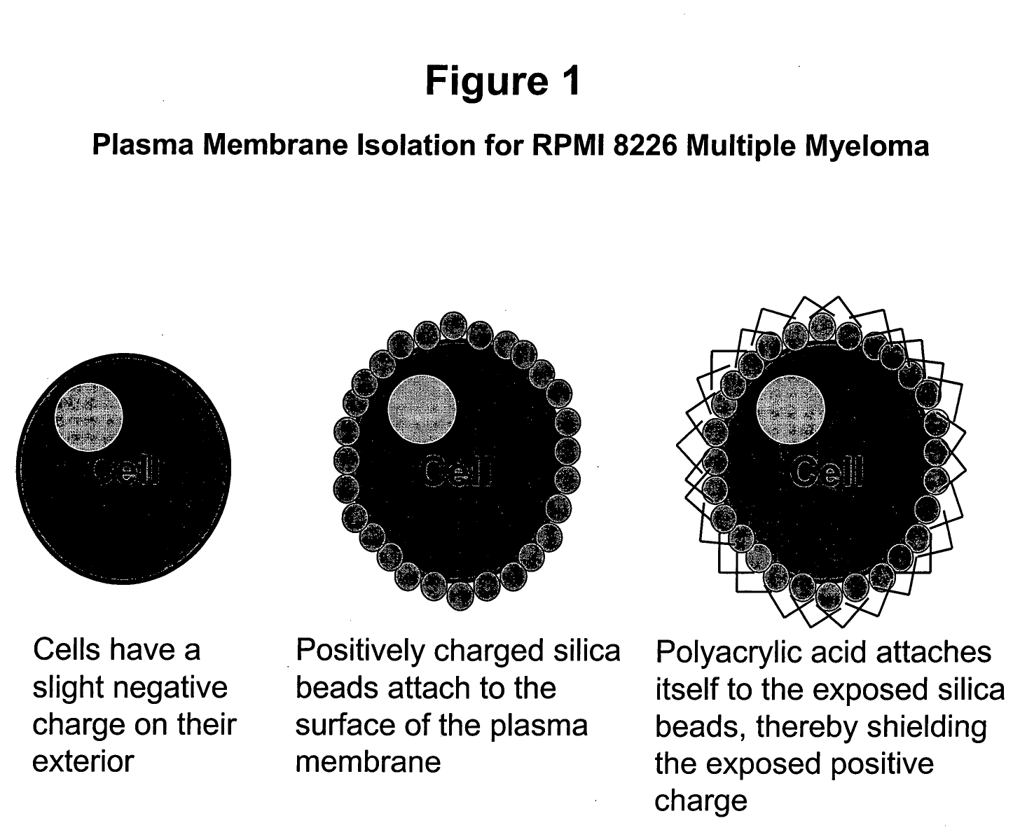

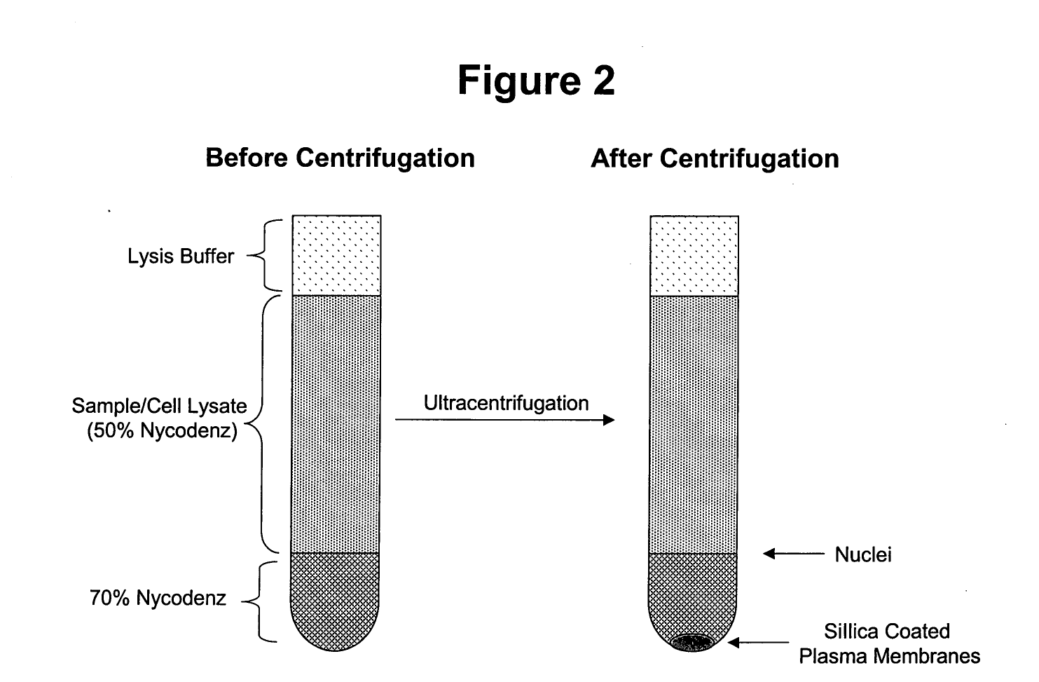

Plasma membranes from RPMI 8226 were isolated using a modified cationic colloidal silica plasma membrane perturbation procedure used for suspension cells (Chaney/Jacobson, Cells a lab manual). Briefly, about 1.5 g wet weight RPMI 8226 cells were washed in plasma membrane coating buffer A (PMCBA-20 mM MES, 150 mM NaCl, 800 mM sorbitol, pH 5.3) and placed drop-wise in a 10% suspension of cationic colloidal silica in PMCBA and placed on ice for 15 min with gentle rocking. The silica-coated cells were washed with PMCBA and placed drop-wise in a solution of 10 mg/ml polyacrylic acid in PMCBA pH 6-6.5 and placed on ice for 15 min with gentle rocking (FIG. 1) The cells were then washed with PMCBA and placed in lysis buffer with protease inhibitors (2.5 mM Imidazole with sigma protease inhibitor cocktail) for 30 minutes to swell the cells. Cells were then lysed using nitrogen cavitation at 1500 psi. The cell lyasate was spun at 900 g for 30 min to sediment nuclei and silica coated plasma membranes. The pellet was resuspended in lysis buffer, diluted with an equal amount of 100% Nycodenz to make a 50% Nycodenz solution and layered over 70% Nycodenz. Lysis buffer was then layered onto each tube and filled to the top (FIG. 2). The tubes were then spun at 60,000 g in an SW60OTi rotor for 23 min. The silica coated plasma membranes pellet to the bottom of the tube leaving the nuclei at the 50%/70% Nycodenz interface. The supernatant was drawn off and the pellets resuspended in lysis buffer. The pellets were washed 3 times in lysis buffer followed by 3 times in 100 mM Na₂CO₃ pH 11.4, with at least 14,000 g spins between each wash. The plasma membranes were then solubilized directly in Laemmli loading buffer, placed in a 60° C. water bath for 15 min, sonicated 5 times for 10 seconds at max setting, and placed in a 60° C. water bath for an additional 15 min and spun at max speed in a micrufuge to pellet silica coating from the solubilized plasma membrane proteins. The plasma membranes were then stored at −80° C.

Preparation of Plasma Membrane Fraction from Cell Culture Monolayers

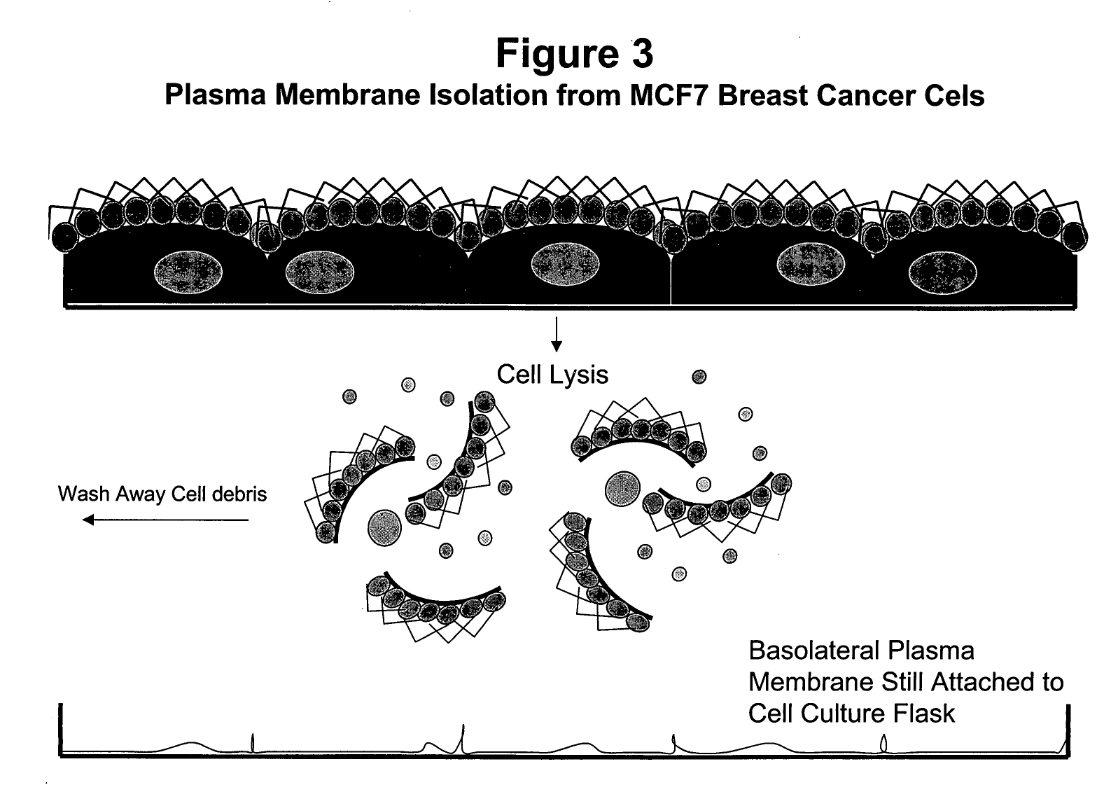

Plasma membranes from MXR MCF-7 cells were isolated using a modified cationic colloidal silica plasma membrane perturbation procedure used for cell culture monolayers (Stolz/Jacobson, Cells a lab manual). In this procedure the cells were coated with- the silica and polyacrylic acid while still attached to the cell culture flask, so that all washes and cell coating solutions were added directly to the flask with the cells still attached. Briefly, MXR MCF-7 cells were grown to confluence in 12 150 cm² cell culture flasks. The media was removed and the cells were washed twice with PBS containing 1 mM MgCl₂, and 1 mM CaCl₂ and then washed with plasma membrane coating buffer B (PMCBB-0.5 mM CaCl₂, 1 mM CaCl₂, 20 mM MES, 135 mM NaCl, pH 5.3). The cells were then coated with a 5% suspension of cationic colloidal silica in PMCBB and left on ice for 1 minute. The silica suspension was removed, followed by a wash with PMCBB to remove excess silica. The cells were then coated with a 10 mg/ml solution of polyacrylic acid in PMCBB pH 6-6.5 and left on ice for 1 minute. The polyacrylic acid solution was removed, followed by a wash with PMCBB to remove any excess polyacrylic acid (FIG. 3). The cells were then washed once quickly with lysis buffer (2.5 mM Imidazole) and then lysis buffer with sigma protease inhibitor cocktail was added to the cell culture flasks and left on ice for 30 min to swell the cells. The flasks were then placed on a bench top and allowed to reach room temp (˜15-30 min). The apical part of the plasma membranes (the part of the plasma membrane coated by the silica and polyacrylic acid) were sheared from rest of the cell by pipetting the lysis buffer in each flask up and down over the cells, or by using a syringe with a long needle to apply enough shearing force to rip off the top part of the plasma membranes. At this point the apical membranes were drawn off with the pipette-aid and were treated as described in the section describing the isolation of plasma membranes from suspension cells following cell lysis, up until the final wash step. Following the removal of the nuclei using the Nycodenz gradient, silica coated plasma membrane pellet was washed 3 times with lysis buffer. The silica coated plasma membrane sheets were then resuspended in 30 ml of 100 mM Na₂CO₃ pH 11.4 in a 50 ml centrifuge tube and placed in a sonication bath for 30-40 minutes with vortexing every 5 minutes. The tube was spun at 300 g for 15 min. The pellet was resuspended in 30 ml of 100 mM Na₂CO₃ pH 11.4 and both samples were spun at 14,000 g in an SW28 rotor for 20 min. Each pellet was resuspended in 1.5 ml of Na₂CO₃ and spun at max speed for 20 min in a microfuge. The apical plasma membrane pellets from both tubes were then solubilized in 2% SDS or directly in Laemml loading buffer and the plasma membrane proteins were solubilized using sonication and a 60° C. water bath as described in the previous section.

At this point the basolateral plasma membranes were still attached to the cell culture flasks. Following cell lysis, the cell culture flasks were washes once quickly with lysis buffer and then lysis buffer with protease inhibitors was added to each flask and incubated for 5 min at room temperature with rocking. The lysis buffer was poured off and 5M NaCl was added to each flask and incubated for 5 min at room temperature with rocking. The 5M NaCl was poured off and PBS containing 10 mM EDTA was added to each flask and incubated for 5 min at room temperature with rocking. The PBS/EDTA solution was poured off and washed once quickly with 100 mM Na₂CO₃, pH 11.4 to remove any excess PBS/EDTA. 100 mM Na₂CO₃, pH 11.4 was added to each flask and incubated for 5 min at room temperature with rocking. The Na₂CO₃ was poured off and replaced with 7 ml 100 mM Na₂CO₃, pH 11.4 per flask. The basolateral plasma membranes were then scraped from the bottom of the cell culture flask using a cell scraper and spun at 14,000 g in an SW28 rotor for 20 min. The purified basloateral plasma membranes pellet to the bottom of the ultracentrifuge tubes and are then resuspended in a 1.5 ml microfuge tube and spun at max speed in a microfuge for 20 min to pellet the plasma membranes. The basolateral plasma membrane pellet can then be solubilized in 2% SDS or directly in Laemml loading buffer and the plasma membrane proteins were solubilized using sonication and a 60° C. water bath as described in the previous section.

1D SDS-PAGE and Western Blot Analysis

75-100 μg of plasma membrane protein was loaded onto 4-15% or 12.5% gels and run according to specifications using the Bio-Rad Criterion precast gel system.

Following electrophoresis, the gels were stained using Bio-Rad Biosafe Coomassie stain. For Western bolt analysis, 25 μg of protein was loaded onto each lane and the proteins were separated by electrophoresis as described above. Following electrophoresis, the proteins on the gel were transferred to a nitrocellulose membrane according to specifications using the Bio-Rad Mini-PROTEAN 3 electrophoresis system and Mini trans-blot cell. Western blotting was done according to specifications using the Sigma ProteoQwestä Colorimetric Western Blotting Kit and the human anti-Na/K ATPase was used as the primary antibody. Following electrophoresis and western blotting, the stained gels and nitrocellulose were scanned using the Bio-Rad GS-800 densitometer. Enrichment of the plasma membrane fractions were quantitated from the western blot images using the ImageQuant image analysis software by Amersham Biosciences.

Mass Spectrometry and Protein Identification

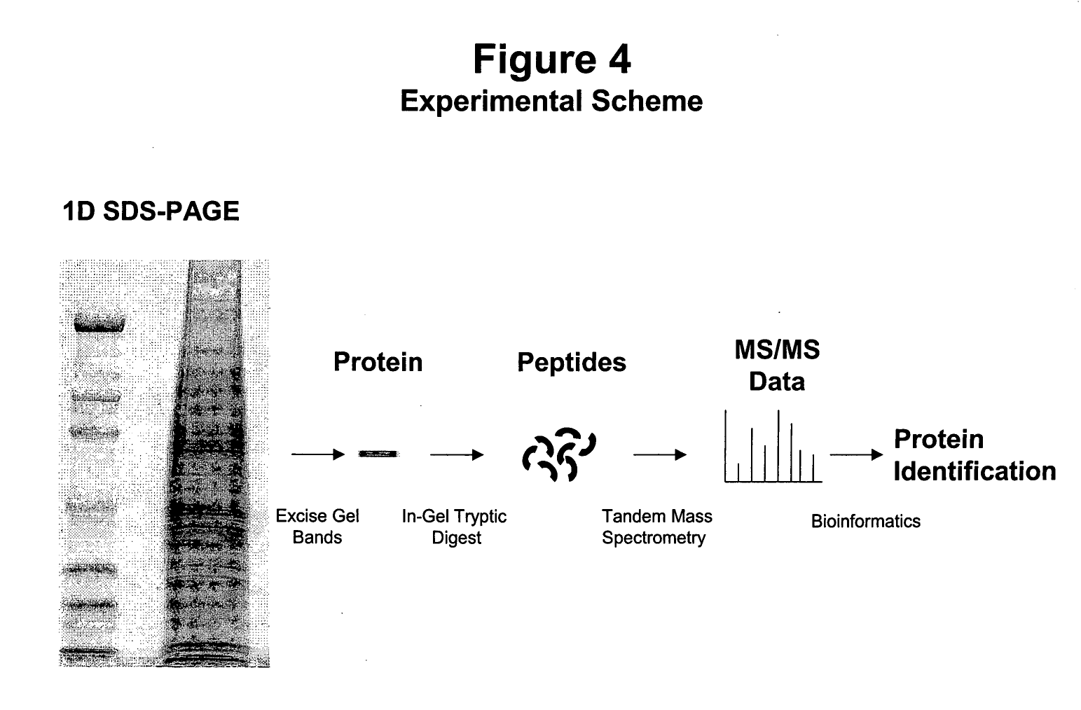

Protein bands were excised from the gel and in-gel tryptic digestion was performed as described by Jensen et al. After extraction from the gel bands, the tryptic peptides were desalted using Zip Tip C18 pipette tips. The acetonitrile/TFA was removed using a speed vac and the peptides were resuspended in a solution of methanol/water/acetic acid (50/50/2) in preparation for MS/MS analysis. Tandem mass spectra for the tryptic peptides were obtained using the Applied Biosystems Qstar Pulsar i, using NanoES spray capillaries and the nanospray ion source from Protana. Protein identification was determined from the data obtained using the MASCOT search engine from Matrix Science and the integrated Qstar software Analyst QS with Bioanalyst. FIG. 4 shows a scheme used for the identification of proteins from the plasma membrane fractions.

Plasma Membrane Enrichment

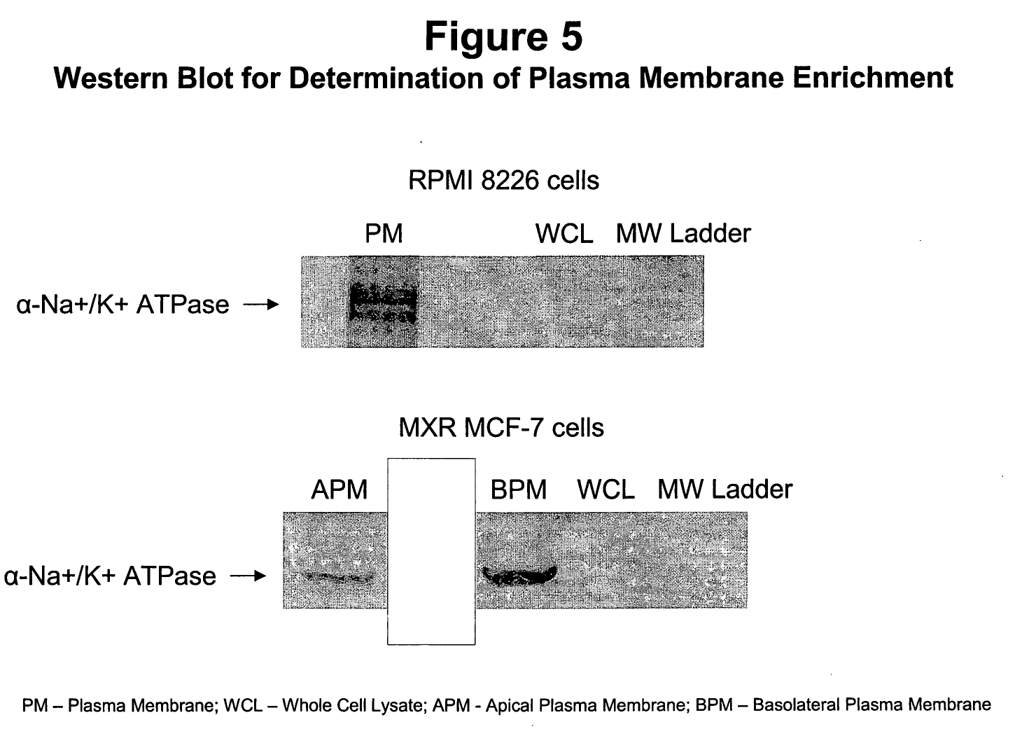

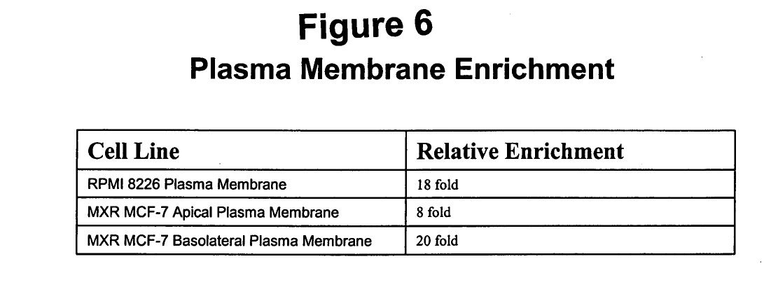

Western blot experiments were performed to determine the enrichment of the plasma membrane fractions isolated using this modified technique using the antibody against human Na+/K+ ATPase which is located in the plasma membrane (FIG. 5). Using the ImageQuant software the relative enrichments of each plasma membrane fraction was determined and the results are in FIG. 6. The high degree of plasma membrane enrichment using this technique allows for the identification of low abundance proteins from the plasma membrane.

Protein Identification

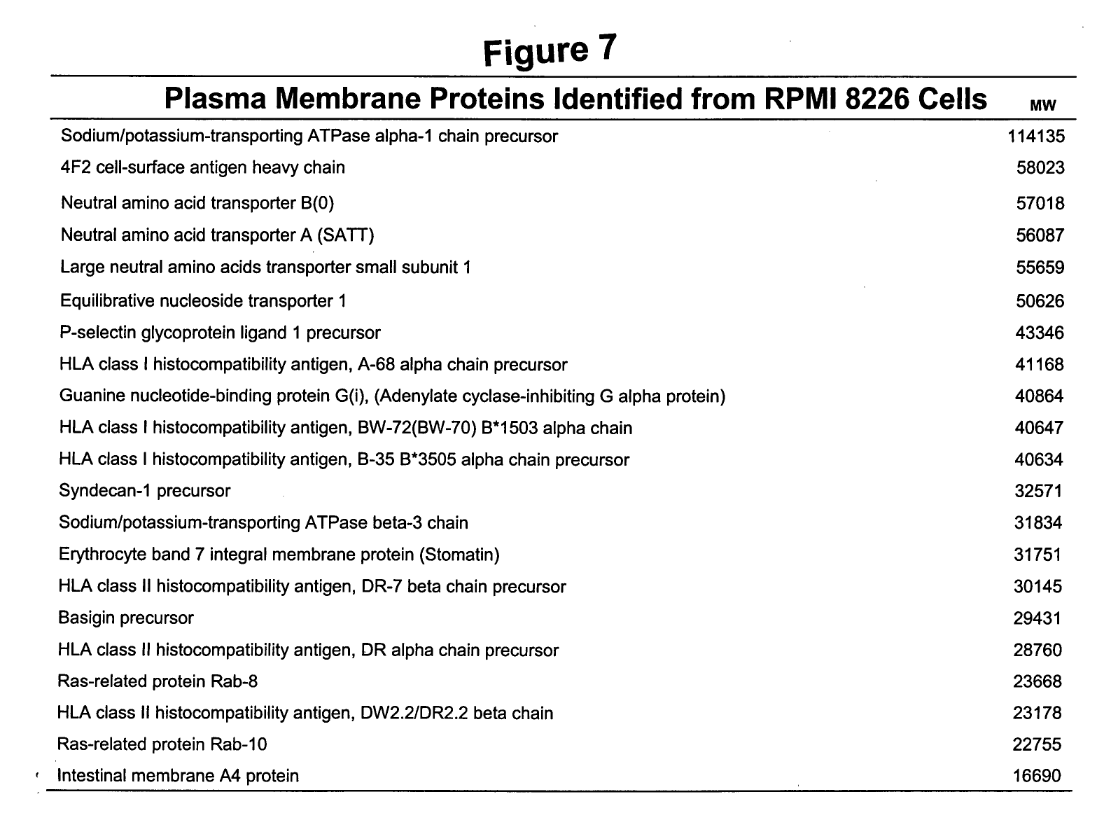

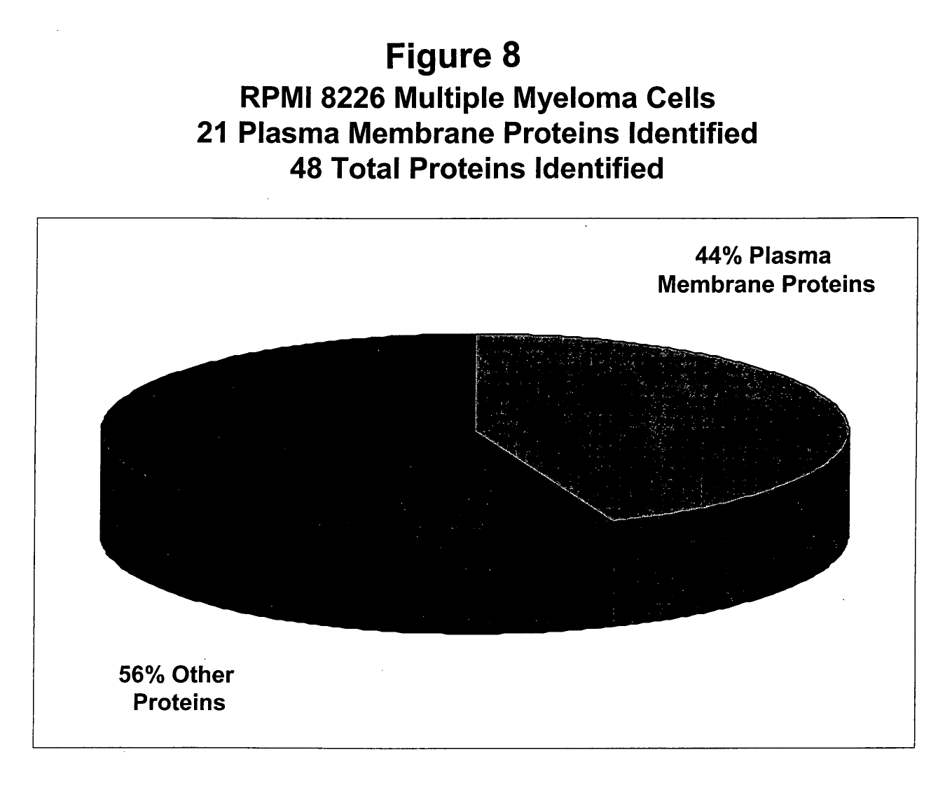

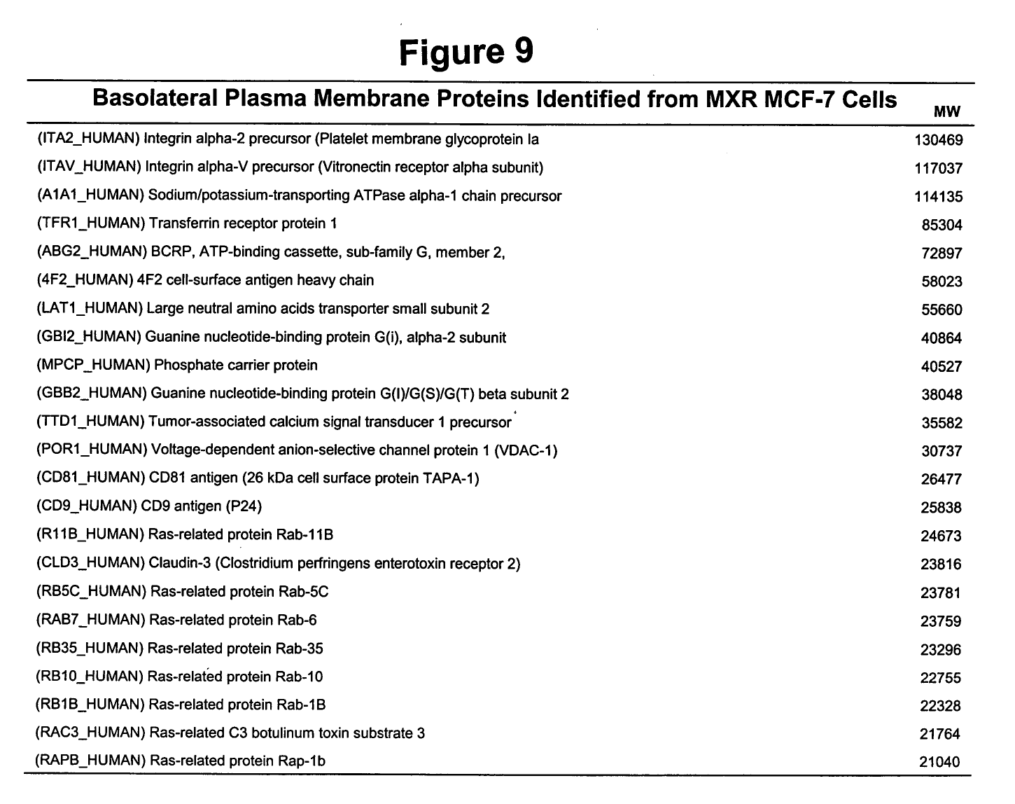

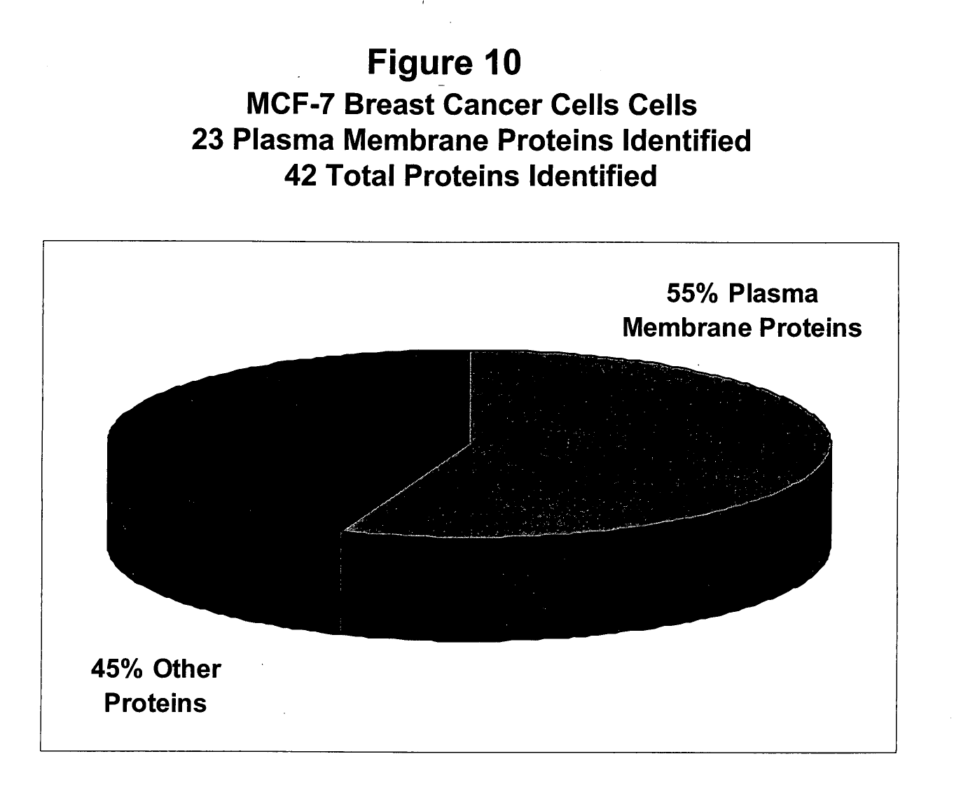

Plasma membrane proteins identified from RPMI 8226 multiple myeloma cells are shown in FIGS. 7 and 8. Plasma membrane proteins identified from MXR MCF-7 breast cancer cells are shown in FIGS. 9 and 10.

1. A method of separating plasma membranes, comprising the steps of:
isolating plasma membranes with a cationic colloidal silica plasma membrane isolation step;
washing the isolated plasma membranes with sodium carbonate to produce silica coated plasma membrane;
prior to any proteolytic digestion, solubilizing the silica coated plasma membrane in 2% SDS;
separating the plasma membrane proteins on an SDS PAGE gel.

2. The method of claim 1, further comprising excising the proteins;
subjecting the proteins to in gel trypsin digestion; and then
analyzing the proteins with mass spectrometry and bioinformatics database searching.

3. A method of separating plasma membranes, comprising the steps of:
isolating plasma membranes;
washing the isolated plasma membranes;
solubilizing the silica coated plasma membrane;
separating the plasma membrane proteins.

4. The method of claim 3, further comprising excising the proteins;
subjecting the proteins to in gel trypsin digestion; and then
analyzing the proteins with mass spectrometry and bioinformatics database searching.

5. The method of claim 3 wherein said washing of the isolated plasma membranes is conducted with sodium carbonate.

6. The method of claim 3 wherein the solubilizing of the silica coated plasma membrane is conducted in 2% SDS.

7. The method of claim 3 wherein the plasma membrane proteins are separated with a cationic colloidal silica plasma membrane.

8. The method of claim 3 wherein the solubilizing of the silica coated plasma membrane is conducted prior to any proteolytic digestion.

9. The method of claim 8, further comprising excising the proteins;
subjecting the proteins to in gel trypsin digestion; and then
analyzing the proteins with mass spectrometry.

10. The method of claim 8 wherein said washing of the isolated plasma membranes is conducted with sodium carbonate.

11. The method of claim 8 wherein the solubilizing of the silica coated plasma membrane is conducted in 2% SDS.

12. The method of claim 3 wherein the solubilizing of the silica coated plasma membrane is conducted prior to any proteolytic digestion.

13. The method of claim 12, further comprising excising the proteins;
subjecting the proteins to in gel trypsin digestion; and then
analyzing the proteins with mass spectrometry.

14. The method of claim 12 wherein said washing of the isolated plasma membranes is conducted with sodium carbonate.

15. The method of claim 12 wherein the solubilizing of the silica coated plasma membrane is conducted in 2%, SDS.
